(19) United States
(12) Patent Application Publication
(10) Pub. No.: US 2008/0281414 A1
(43) Pub. Date: Nov. 13, 2008

(54) Supplementary Intraocular Lens

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/106,182

(22) Filed: Apr. 18, 2008

Related U.S. Application Data
(60) Provisional application No. 60/912,884, filed on Apr. 19, 2007.

(51) Int. Cl. A61F 2/16

(52) U.S. Cl. 623/6.17; 623/6.32; 623/6.23; 623/6.27

(57) ABSTRACT

A method is disclosed to place supplemental IOLs in the anterior chamber, posterior chamber or both, in combination with the eye's natural lens or a separate, implanted artificial lens.

### FIELD OF THE INVENTION

This invention relates to implantable intraocular lenses, and more particularly, to an intraocular lens that can be implanted to complement an already-implanted intraocular lens for purposes of improving accommodation or providing UV and blue light protection.

### BACKGROUND OF THE INVENTION

In U.S. Pat. No. 5,470,932, the patentee describes the effect of changes in the structure of the human eye that result in increased sensitivity to ultraviolet light and blue light. There are two circumstances where these factors become important. The first is where the natural lens in the eye is intact, but has undergone structural or chemical changes over time. The second occurs when the natural lens has been replaced by an artificial intraocular lens (IOL) which may lack structure to provide protection or accommodative capacity.

As stated in the '932 patent:

The assessment of optical hazards in recent years has led to the recognition of the possible hazards to the retina associated with blue light (400-500 nm). If the blue light hazard is a real threat to vision, then the UV/visible transmission characteristics of ophthalmic lenses and intraocular lenses (IOLs) in particular, should be modified to provide adequate protection from blue light hazards encountered in the environment.

In the ambient environment, solar radiation is the primary hazard division. The sun freely emits UV (visible and IR) radiation much of which is absorbed by the atmosphere. The solar radiation that is transmitted through the atmosphere and reaches the earth's surface consists of UV-B radiation (230-300 nm) near UV or UV-A radiation (300-400 nm) visible lights (400-700 nm) and near IR radiation (700-1400 nm). The ocular media of man in its normal healthy state freely transmits near IR and most of the visible spectrum to the retina but UV-B radiation is absorbed by the cornea and does not reach the retina. The transmission of near UV and the blue portion of the visible spectrum can be absorbed by the crystalline lens depending on age.

The human crystalline lens changes its UV invisible transmission characteristics as it ages. In infancy the human lens will freely transmit near UV invisible light above 300 nm, but with further aging the action of UV radiation from the environment causes the production of yellow pigments, fluorogens, within the lens. By age 54, the lens will not transmit light below 400 nm and the transmission of light between 400 and 500 nm is greatly diminished. As the lens ages, it continuously develops a yellow color, increasing its capacity to filter out near UV and blue light. Therefore, after cataract removal, the natural protection provided by the aged human lens is also removed. If the cataract is replaced by an IOL, usually UV protection is provided, but blue light protection is still lacking.”

The '932 patent (Jinkerson) together with U.S. Pat. No.5,543,504 (Jinkerson); U.S. Pat. No. 5,528,322 (Jinkerson); and U.S. Pat. No. 5,662,707 (Jinkerson) all teach and describe polymerizable yellow dyes and their use in ophthalmic lenses. The use of such dyes in the manufacturing of IOLs replaces the protection afforded by the natural lens as it ages.

U.S. Pat. No. 6,926,736 (Peng et al) teaches and describes an accommodative intraocular lens comprising separate interior and posterior optics with the posterior optic serving as a holder for the anterior optic and with the anterior optic being designed to be moved by the action of the ciliary muscle.

U.S. Pat. No. 6,899,732 (Zadno-Azizi et al) teaches and describes a method of implanting an intraocular lens system wherein the lens system has anterior and posterior optic elements separated by translation members which allow the distance between the anterior and posterior optics to be changed when force is applied by the action of the ciliary muscle.

U.S. Pat. No. 6,824,563 (Lang) teaches and describes binocular lens systems consisting of two lenses of differing optical characteristics with each lens being implanted into one eye of a patient. The lenses may be intraocular lenses, contact lenses or corneal inlays.

U.S. Pat. No. 6,527,389 (Portney) teaches and describes a multifocal ophthalmic lens which may be either a contact lens or an IOL with each lens having a series of concentric alternating “power zones” providing continuous variation without abrupt changes or edges.

U.S. Pat. No. 4,512,040 (McClure) teaches and describes a bifocal intraocular lens having a fluid reservoir and a fluid chamber with the chamber filling or draining in response to a change in the inclination of a person's head and thereby line of sight wherein the presence or absence of fluid in the chamber changes the optical characteristics of the lens.

U.S. Pat. No. 6,616,692 (Glick, et al.) teaches and describes an intraocular lens combination with one IOL designed to provide an accommodation feature while the other provides a selected optical power to aid in accommodation.

Use of tinted optical elements and use of multiple optical elements in a single construction are well represented in the prior art. The teachings of the prior art presume that the particular lens or lens system selected for implantation meets the needs of the patient as of the time the implantation takes place.

The present invention addresses the situation that occurs when the optical needs of a patient change after an implantation has already occurred. For example, once an IOL is implanted into the posterior chamber, it is very difficult to remove. Removal of a natural lens is facilitated by the fact that the lens may be chopped or split and then emulsified with the particles being aspirated from the eye through a very small incision. An artificial IOL can be implanted through an equally small incision by being folded and injected through the incision into the posterior chamber. The IOL is then allowed to unfold and is held in place by haptics which contact the walls of the posterior chamber. Once such an artificial IOL is implanted, it is very difficult to remove because the plastic materials commonly selected to manufacture such IOLs are difficult to fracture or break up. This means that the removal of an already implanted IOL calls for more extreme surgical efforts commonly requiring incisions of much greater size. If at all possible, the removal of an implanted artificial IOL is thus preferably avoided.

Under different circumstances, a patient may desire additional protection from, for example, UV rays or blue light. This may occur even though the patient has not had an artificial IOL implanted but has retained the use of his natural lenses. The present invention addresses these situations by providing an additional IOL, tinted to provide UV or blue light protection and which can be inserted into the eye whether or not the patient's natural lens or an implantable IOL is present.

In addition to providing UW/blue light protection, a second implant can also be used to correct for errors present in the original lens or to accommodate other changes in the patient's visions such as presbyopeia, astigmatisms and the like.

To accomplish these objects, the present invention provides for the implantation of an IOL into the anterior chamber of the eye or into the sulcus in front of an existing natural lens or artificial IOL.

### SUMMARY OF THE INVENTION

Methods are provided for the selection and implantation of a supplementary corrective or protective IOL with the steps being dependent upon the existing condition of the eye. For an eye which has retained its natural lens but which requires correction or UV/blue light protection, the supplementary lens may be implanted in the anterior chamber or in the sulcus of the posterior chamber in front of the existing natural lens.

### BRIEF DESCRIPTION OF THE DRAWINGS

These and further aspects of the present invention will be apparent upon complete reading of the specification and claims read in connection with the attached drawings, wherein:

FIG. 1 is a lateral schematic view of a human eye;

FIG. 2 shows the eye of FIG. 1 and the positioning of a supplementary lens in the anterior chamber and in the posterior chamber; and

FIG. 3 shows the eye of FIG. 1 and the positioning of a supplemental lens in the posterior chamber and two supplementary lenses in the anterior chamber.

### DETAILED DESCRIPTION OF THE DRAWINGS

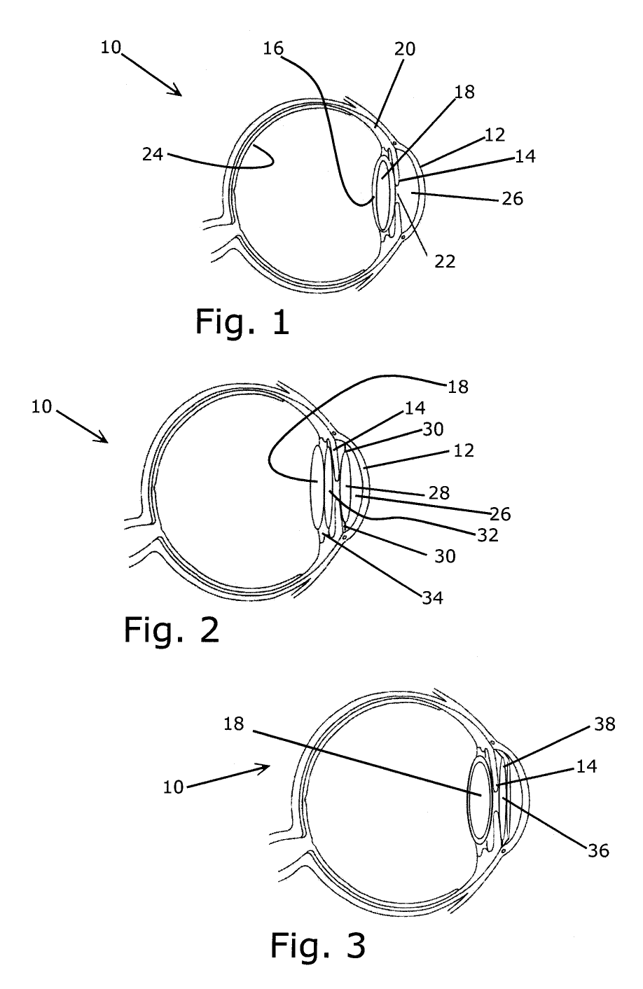

Referring now to FIG. 1, the numeral 10 identifies a schematic drawing of the human eye having a cornea 12, iris 14, capsular bag 16, lens 18, ciliary muscle 20 and pupil 22. At the rear of the eye is the retina 24 and the space between cornea 12 and iris 14 is the anterior chamber 26.

Lens 18 represents either the eye's natural lens or an artificial IOL as both would be positioned within capsular bag 16. For the purposes of the following descriptions, the term “principal lens” shall refer to either the natural lens or an IOL inserted to replace the natural lens.

Referring now to FIG. 2, the numeral 28 identifies a first supplementary lens positioned within anterior chamber 26 and in front of principal lens 18. A pair of haptics 30 hold lens 28 in place after it has been implanted.

When the anterior chamber 26 is selected as the site for the implantation of supplementary lens 28, principal lens 18 can remain undisturbed. This is a significant advantage where principal lens 18 is an artificial IOL, the removal of which would be extremely difficult. Artificial IOLs are not susceptible of being emulsified using phacoemulsification techniques as are natural lenses, so it is preferable to leave principal lens 18 in place whenever possible.

Numeral 32 identifies a second supplementary lens positioned behind iris 14 and in front of principal lens 18 within that portion of the eye identified as the sulcus 34.

When natural lens 18 is to be removed and replaced with an artificial IOL, the surgeon may implant an artificial IOL 18 and a second supplementary lens 32 within sulcus 34 in order to impart to the eye the desired protective or corrective effect.

Both first supplementary lens 28, second supplementary lens 32 are of a well-known type that can be folded and implanted through a relatively small incision made in the cornea.

It is contemplated that the implantation of supplementary lens 28 will be done at a time separate from the replacement of natural lens 18 with an artificial IOL. Indeed, first supplementary lens 28 can be installed before any replacement of natural lens 18 is made or required.

The characteristics of supplementary lens 28 can be selected as needed or desired. For example, lens 28 can be tinted to provide necessary or desired UV or blue light protection. Lens 28 can also employ accommodative features such as those shown in the prior art in order to further correct or improve the patient's vision.

Referring now to FIG. 3, there are also advantages to positioning a supplemental IOL 36 in the anterior chamber along with a second lens 38. For example, a tinted lens 38 can be implanted in the anterior chamber in combination with a specialized IOL 36 such as a toric design (to correct astigmatism), a multifocal design (to correct presbyopia), an aspherical design (to improve image quality) or a monofocal design. The choice of lens types and placement will be the surgeon's decision based upon the patient's requirements.

While the foregoing describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. A method for providing vision adjustments and protection to a principal lens present in the posterior chamber of a mammalian eye, said principal lens being a natural lens or an artificial intraocular lens (IOL), said method comprising the steps of:
determining the type of adjustment or protection needed for said principal lens;
providing at least one supplementary lens selected to provide the desired adjustment or correction; and
inserting said at least one supplemental lens into the posterior chamber proximate to said principal eye.

2. The method of claim 1 wherein said at least one supplementary lens is tinted to provide protection from ultraviolet radiation.

3. The method of claim 2 wherein said tint is yellow.

4. The method of claim 1 further comprising the step of inserting a second supplemental lens in the anterior chamber of said eye.

5. The method of claim 4 wherein said second supplemental lens is a multifocal lens.

6. The apparatus as recited in claim 4 wherein said second supplemental lens is a toric lens.

7. The method of claim 4 wherein said second supplemental lens is an aspherical lens.

8. A method for providing vision adjustments and protection to a principal lens present in the posterior chamber of a mammalian eye, said principal lens being a natural lens or an artificial intraocular lens (IOL), said method comprising the steps of:
determining the type of adjustment or protection needed for said principal lens;
providing at least one supplementary lens selected to provide the desired adjustment or correction; and
inserting said at least one supplemental lens into the anterior chamber proximate to said principal eye.

9. The method of claim 8 wherein said at least one supplementary lens is tinted to provide protection from ultraviolet radiation.

10. The method of claim 9 wherein said tint is yellow.

11. The method of claim 8 further comprising the step of inserting a second supplemental lens in the anterior chamber of said eye.

12. The method of claim 8 wherein said second supplemental lens is a multifocal lens.

13. The apparatus as recited in claim 8 wherein said second supplemental lens is a toric lens.

14. The method of claim 8 wherein said second supplemental lens is an aspherical lens.
